(19) United States
(12) Patent Application Publication
(10) Pub. No.: US 2023/0086479 A1
(43) Pub. Date: Mar. 23, 2023

(54) FLUORESCENT SILICA NANOPARTICLES AND METHOD FOR MANUFACTURING FLUORESCENT SILICA NANOPARTICLES

(71) Applicant: Konica Minolta, Inc., Tokyo (JP)

(72) Inventors: Tomohiro KUDO, Hino-shi, Tokyo (JP); Takato CHIBA, Hachioji-shi, Tokyo (JP)

(21) Appl. No.: 17/795,143

(22) PCT Filed: Jan. 29, 2021
(86) PCT No.: PCT/JP2021/003228

§ 371 Date: Jul. 25, 2022

(30) Foreign Application Priority Data
Feb. 3, 2020 (JP) 2020-016145

(51) Int. Cl. C09K 11/02; C09K 11/06; C09B 5/62; A61K 49/00

(52) U.S. Cl. CPC C09K 11/02; C09K 11/06; C09B 5/62; A61K 49/005; C09K 2211/1022; C09K 2211/1014; C09K 2211/1007; G01N 21/64

(57) ABSTRACT

The present invention relates to providing fluorescent silica nanoparticles having high luminance even when many fluorescent dyes are contained in silica particles. Fluorescent silica nanoparticles according to the present invention is fluorescent silica nanoparticles including silica nanoparticles and fluorescent dyes contained in the silica nanoparticles, in which a total volume of the fluorescent dyes is 5% or more with respect to a total volume of the fluorescent silica nanoparticles, and an emission quantum yield of the fluorescent silica nanoparticles is 10% or more.

### TECHNICAL FIELD

The present invention relates to fluorescent silica nanoparticles and method for manufacturing the fluorescent silica nanoparticles.

### BACKGROUND ART

In recent years, a molecular imaging technique has attracted high attention in clinical fields and basic research. Molecular imaging is a technique for visualizing movement of molecules in a living body, which has not been able to be visualized so far. For example, the molecular imaging is widely used for various purposes such as analysis of biomolecules at a molecular level, research on dynamics of viruses and bacteria that cause diseases, and evaluation of effects and the like of drugs on living bodies. In particular, fluorescence imaging performed using a fluorescent substance is widely used for detection of a trace substance in a living body, due to its excellent detection sensitivity, operability, and the like.

In diagnosis and research using the fluorescence imaging, there has been proposed a method of detecting fluorescence of a labeling reagent with high sensitivity by bonding a fluorescent substance as a labeling reagent to a biological substance desired to be detected, and irradiating with predetermined excitation light. By a fluorescent signal obtained by such fluorescence imaging, there is required a fluorescent labeling material having characteristics for performing quantification of biomolecule interaction, kinetic observation of biomolecules over a long period of time, ultra-high sensitivity observation, and the like.

For example, Patent Literature 1 discloses a method for obtaining highly sensitive fluorescent nanoparticles by hydrolyzing, together with a tetraalkoxysilane, an organosiloxane compound to which a fluorescent dye molecule is bonded, to obtain a core of a fluorescent dye molecule-containing silica particle, and further adding tetraalkoxysilane to form a shell covering the core of the silica particle.

### CITATION LIST

### Patent Literature

Patent Literature 1: JP 2009-221059 A

### SUMMARY OF INVENTION

### Technical Problem

The present inventors have attempted to obtain fluorescent silica nanoparticles with higher luminance by causing the silica particles to internally contain more fluorescent dyes, in fluorescent silica nanoparticles obtained by hydrolysis and polycondensation of a fluorescent dye molecule having an alkoxysilyl group and alkoxysilane, as described in Patent Literature 1. However, even if many fluorescent dyes are contained in the silica particles, it has not been possible to obtain silica nanoparticles with high luminance.

The present inventors have intensively studied the reason why it has not been possible to obtain silica nanoparticles with high luminance, and considered that, when many fluorescent dyes are contained in silica particles, concentration quenching occurs, and an emission quantum yield decreases, so that high-luminance silica nanoparticles cannot be obtained. Here, the concentration quenching refers to a phenomenon in which an emission intensity (emission efficiency) of a phosphor decreases as a concentration of an emission center ion increases in the phosphor. As one of the causes of this, it is conceivable that a process occurs in which excitation energy moves between emission center ions, and then is non-radiationally consumed without light emission.

The present invention has been made in view of the above circumstances, and an object is to provide fluorescent silica nanoparticles having high luminance even if many fluorescent dyes are contained in silica particles. Further, an object of the present invention is to provide a method for manufacturing the fluorescent silica nanoparticles.

### Solution to Problem

Fluorescent silica nanoparticles according to an embodiment of the present invention is fluorescent silica nanoparticles including silica nanoparticles and fluorescent dyes contained in the silica nanoparticles, in which a total volume of the fluorescent dyes is 5% or more with respect to a total volume of the fluorescent silica nanoparticles, and an emission quantum yield of the fluorescent silica nanoparticles is 10% or more.

Further, a method for manufacturing fluorescent silica nanoparticles according to an embodiment of the present invention is a method for manufacturing the fluorescent silica nanoparticles described above. The method includes a step of continuously adding alkoxysilane to liquid containing fluorescent dyes, ammonia, and water so that a molar ratio of the fluorescent dyes to the alkoxysilane is in a range of 1 to 30.

### Advantageous Effects of Invention

According to the present invention, it is possible to provide fluorescent silica nanoparticles containing many fluorescent dyes in silica particles and having high luminance. In addition, the present invention can provide a method for manufacturing the fluorescent silica nanoparticles.

### DESCRIPTION OF EMBODIMENTS

[Fluorescent Silica Nanoparticles]

Fluorescent silica nanoparticles according to an embodiment of the present invention is fluorescent silica nanoparticles including silica nanoparticles and fluorescent dyes contained in the silica nanoparticles, in which a total volume of the fluorescent dyes is 5 vol% or more with respect to a total volume of the fluorescent silica nanoparticles, and an emission quantum yield of the fluorescent silica nanoparticles is 10% or more.

(Silica Nanoparticles)

The fluorescent silica nanoparticles according to the present embodiment contain silica nanoparticles as a base material.

The silica nanoparticle is not particularly limited as long as a fluorescent dye with a physical or chemical bonding force can be contained. The silica nanoparticle is, for example, a polymer obtained by hydrolyzing and polycondensing alkoxysilane.

Examples of the alkoxysilane include tetraalkoxysilane, trialkoxysilane, dialkoxysilane, and the like. Among them, tetraalkoxysilane or trialkoxysilane is preferable, and tetraalkoxysilane is more preferable.

Examples of the tetraalkoxysilane include tetraethoxysilane (TEOS), tetramethoxysilane, tetrabutoxysilane, tetrapropoxysilane, tetraisopropoxysilane, tetrakis(2-ethylhexyloxy)silane, and the like. Among them, tetraethoxysilane (TEOS) is particularly preferable.

Examples of the trialkoxysilane include triethoxy-3-aminopropylsilane, N-(2-aminoethyl) aminopropyltrimethoxysilane, allyltriethoxysilane, trimethoxyallylsilane, 3-(acryloxy)propyltrimethoxysilane, N-(3-triethoxysilylpropyl)ethienediane, (11-azidoundecyl)trimethoxysilane, N-[3-(trimethoxysilyl)propyl]hexamethylenediamine, bis[3-(trimethoxysilyl)propyl]amine, ethylenebis(trimethoxysilane), 5-(triethoxysilyl)-2-norbornene, and benzyltriethoxysilane.

(Fluorescent Dye)

The fluorescent silica nanoparticles according to the present embodiment include fluorescent dyes contained in the silica nanoparticles.

The fluorescent dye is not particularly limited as long as fluorescence can be emitted. Examples of the fluorescent dye include TAMRA, rhodamine 6G, fluorescein, perylene, Alexa, cyanine, pyrene, Solvent Yellow, and the like, or derivatives of these, and the like.

A content of the fluorescent dyes is preferably large in order to cause the fluorescent silica nanoparticles to have high luminance. From this viewpoint, the content of the fluorescent dyes is preferably 5 vol % or more with respect to a total volume of the fluorescent silica nanoparticles, further preferably 10 vol % or more, further preferably 20 vol % or more, further preferably 30 vol % or more, and further preferably 40 vol % or more.

Whereas, when the content of the fluorescent dyes is too large, silica nanoparticles cannot be formed even if alkoxysilane is hydrolyzed and polycondensed. From this viewpoint, the content of the fluorescent dyes is preferably 70 vol % or less with respect to the total volume of the fluorescent silica nanoparticles.

Further, the fluorescent dye preferably has an alkoxysilyl group. When the fluorescent dye has an alkoxysilyl group, the alkoxysilyl group and alkoxysilane forming the silica nanoparticles are bonded, and the fluorescent dye can be easily contained into the silica nanoparticles.

The fluorescent dye and the alkoxysilyl group may be simply bonded via, for example, an ester bond, a peptide bond, or the like.

The fluorescent dye having the alkoxysilyl group can be obtained, for example, by causing reaction between a fluorescent dye having an NHS ester group with 3-aminopropyltriethoxysilane (APS). Examples of the fluorescent dyes having the NHS ester group include 5-Carboxy TAMRA-NHS ester, 5-Carboxyrhodamine 6G-NHS ester, 5-Carboxyfluorescein-NHS ester (manufactured by Invitrogen), Carboxyfluorescein-PEG12-NHS, Fluorescein-PEG6-NHS ester, Fluorescein-PEG6-bis-NHS ester, BDP FL-NHS ester, Cy3 NHS ester, monoSulfo-Cy3 NHS ester, Cy3.5 NHS ester, Cy5-NHS ester, monoSulfo-Cy5 NHS ester, diSulfo-Cy5 NHS ester, Cy5-PEG6-NHS ester, Cy5.5 NHS ester, Cy7 NHS ester, Cy7.5 NHS ester, Sulfo-Cy3 NHS ester, Sulfo-Cy5 NHS ester, Sulfo-Cy7 NHS ester, solvent yellow98 NHS ester, and the like.

(Average Particle Diameter and Coefficient of Variation)

In addition, when an average particle diameter of the fluorescent silica nanoparticles is too large, it is difficult to stain proteins expressed in gaps between cells or the like. Therefore, the average particle diameter is preferably 250 nm or less, more preferably 150 nm or less, and still more preferably 100 nm or less. A lower limit of the average particle diameter of the fluorescent silica nanoparticles is not particularly limited, but is, for example, preferably 5 nm or more, and more preferably 10 nm or more. Note that the average particle diameter can be determined by measuring a major axis of each particle (100 pieces or more) shown in an image captured with a scanning electron microscope and taking an average value thereof.

The coefficient of variation in particle diameter of the fluorescent silica nanoparticles is preferably small. When the coefficient of variation is small, sizes of the particles become uniform, and fluorescence imaging can be performed with constant luminance. From the above viewpoint, the coefficient of variation in particle diameter of the fluorescent silica nano nanoparticles is preferably 20% or less, and more preferably 15% or less. A lower limit of the coefficient of variation is not particularly limited, but can be, for example, 1% or more.

(Atomic Number Ratio Between Carbon and Silicon)

In the fluorescent silica nanoparticles according to the present embodiment, C/Si, which is an atomic number ratio of carbon to silicon on a surface of the fluorescent silica nanoparticles, is relatively small Here, C and Si represent the abundance of the fluorescent dyes and silica, respectively, and the relatively small C/Si indicates that the fluorescent dyes are not localized on the surface of the fluorescent silica nanoparticles. It is considered that, when the fluorescent dyes are dispersed without being localized in this manner, concentration quenching is suppressed and the emission quantum yield is increased. Conversely, it is considered that, when the C/Si is high, the fluorescent dyes are localized on the surface of the fluorescent silica nanoparticles, so that concentration quenching is likely to occur and the emission quantum yield becomes low. From this viewpoint, C/Si on the surface of the fluorescent silica nanoparticles is preferably relatively low. However, if the C/Si is too low, it is considered that an amount of the fluorescent dyes contained in the fluorescent silica nanoparticles is too small and the luminance is low, so that it is also desired that C/Si is high to some extent.

From the above viewpoint, in the fluorescent silica nanoparticles according to the present embodiment, C/Si, which is an atomic number ratio of carbon to silicon on the surface of the fluorescent silica nanoparticles measured by X-ray photoelectron spectroscopy, is preferably 2 to 10, more preferably 3 to 7, and still more preferably 4 to 5.

(Emission Quantum Yield)

As described above, the fluorescent silica nanoparticles contain as many fluorescent dyes as 5 vol % or more with respect to the total volume of the fluorescent silica nanoparticles, and have a high emission quantum yield of 10% or more. This is considered to be because, the fluorescent silica nanoparticles according to the present embodiment are manufactured by a method for manufacturing fluorescent silica nanoparticles according to an embodiment of the present invention described later, which suppresses localization of the fluorescent dyes (approximation between the fluorescent dyes) and suppresses concentration quenching while many fluorescent dyes are contained.

In the fluorescent silica nanoparticles according to the present embodiment, the emission quantum yield is 10% or more as described above, but more preferably 15% or more, and still more preferably 20% or more. An upper limit of the emission quantum yield is not particularly limited, but can be, for example, 50% or less.

(Luminance)

The fluorescent silica nanoparticles according to the present embodiment have high luminance because concentration quenching is suppressed while many fluorescent dyes are contained. The luminance is preferably high from the viewpoint of enabling measurement to be performed with high sensitivity. Specifically, the luminance of the fluorescent silica nanoparticles according to the present embodiment is preferably 80 or more, more preferably 150 or more, still more preferably 300 or more, and still more preferably 500 or more. An upper limit of the luminance is not particularly limited, but can be, for example, 800 or less.

The method for manufacturing fluorescent silica nanoparticles according to the present embodiment is not particularly limited. For example, the fluorescent silica nanoparticles according to the present embodiment can be manufactured by a manufacturing method described below.

[Method for Manufacturing Fluorescent Silica Nanoparticles]

The method for manufacturing fluorescent silica nanoparticles according to the present embodiment includes a step of continuously adding alkoxysilane to liquid containing fluorescent dyes, ammonia, and water so that a molar ratio of the fluorescent dyes to the alkoxysilane is in a range of 1 to 30.

Here, the continuously adding means adding at least 80% or more of a total addition amount of the alkoxysilane to a reaction solution to which the fluorescent dyes have been added in advance, at a time interval of less than 30 minutes (without a time interval of 30 minutes or more).

According to the manufacturing method described above, it is possible to manufacture fluorescent silica nanoparticles having a high emission quantum yield of 10% or more, in which concentration quenching is suppressed even though many fluorescent dyes of a total volume as large as 5 vol % or more are contained with respect to a total volume of the fluorescent silica nanoparticles. The reason is considered as follows, but is not limited to this.

First, as a premise, it is considered that alkoxysilane is hydrolyzed and polycondensed faster in alkoxysilane and a fluorescent dye (having alkoxysilane group). Then, it is considered that, when the alkoxysilane is continuously added to a solvent containing fluorescent dyes, ammonia, and water as described above, the fluorescent dyes are hydrolyzed, and the alkoxysilane having fast reaction is supplied, so that the fluorescent dyes are prevented from being polycondensed to approximate each other. Conversely, if tetraalkoxysilane is present in the solvent in advance, it is considered that the fluorescent dyes having an alkoxysilyl group are to be added to the solvent in a state in which there is the tetraalkoxysilane that is hydrolyzed to some extent and polycondensed, so that the fluorescent dyes are likely to be localized in the tetraalkoxysilane, and as a result, concentration quenching occurs.

In the method for manufacturing fluorescent silica nanoparticles according to the present embodiment, an addition time of the alkoxysilane is preferably 72 hours or less, more preferably 50 hours or less, still more preferably 1 hour or less, and still more preferably 1 minute or less. When the addition time is short, an average particle diameter of the fluorescent silica nanoparticles decreases, and a CV value of the average particle diameter decreases, so that variation in particle diameter is suppressed.

Note that, here, the addition time refers to a time from a start of addition of the alkoxysilane to an end of addition of a total amount.

Further, a charged molar ratio of the fluorescent dyes to the alkoxysilane (fluorescent dye/alkoxysilane) is preferably large in order to obtain a sufficient contained amount of the fluorescent dyes. However, if the molar ratio is too large, particles cannot be formed. From the above viewpoint. The charged molar ratio (fluorescent dye/alkoxysilane) is preferably 1 to 30, and more preferably 8 to 15.

### EXAMPLES

Hereinafter, the invention according to the present embodiment will be described in detail with reference to Examples, but the invention according to the present embodiment is not limited to these Examples.

[Preparation of Fluorescent Silica Nanoparticles]

### Example 1

<Preparation of Fluorescent Dye>

A perylene dye derivative was prepared according to a synthesis procedure shown below. Propionic acid was added to 1,6,7,12-tetrachloroperylene tetracarboxylic dianhydride (manufactured by FUJIFILM Wako Pure Chemical Corporation, product number: W01COBQA-7294), and refluxed in a solvent for three hours to react (yield 80%). Next, the obtained compound was dissolved in N-methylpyrrolidone (NMP), added with phenol, and caused to react at 80° C. for six hours in the presence of potassium carbonate (K₂CO₃). Next, 3-bromophenol was added, and the compound was caused to react at 120° C. for 16 hours.

Next, the obtained compound was dissolved in 1,4-dioxane, added with 4-pinacolborane phenylacetic acid ethyl, and caused to react at 100° C. for two hours in the presence of bis(dibenzylideneacetone)palladium (pd(dba)₂) and potassium phosphate (K₃PO₄). (Yield 20%).

Next, the obtained compound was dissolved in dioxane, added with an aqueous solution of sodium hydroxide, and caused to react at 60° C. for two hours (yield 73%). Next, the obtained compound was dissolved in tetrahydrofuran (THF), added with 1-(3-Dimethylaminopropyl)-3-ethylcarbodiimide (EDC) and N-hydroxysuccinimide, and caused to reach at 40° C. for four hours (yield 76%). In this way, the perylene dye derivative was obtained.

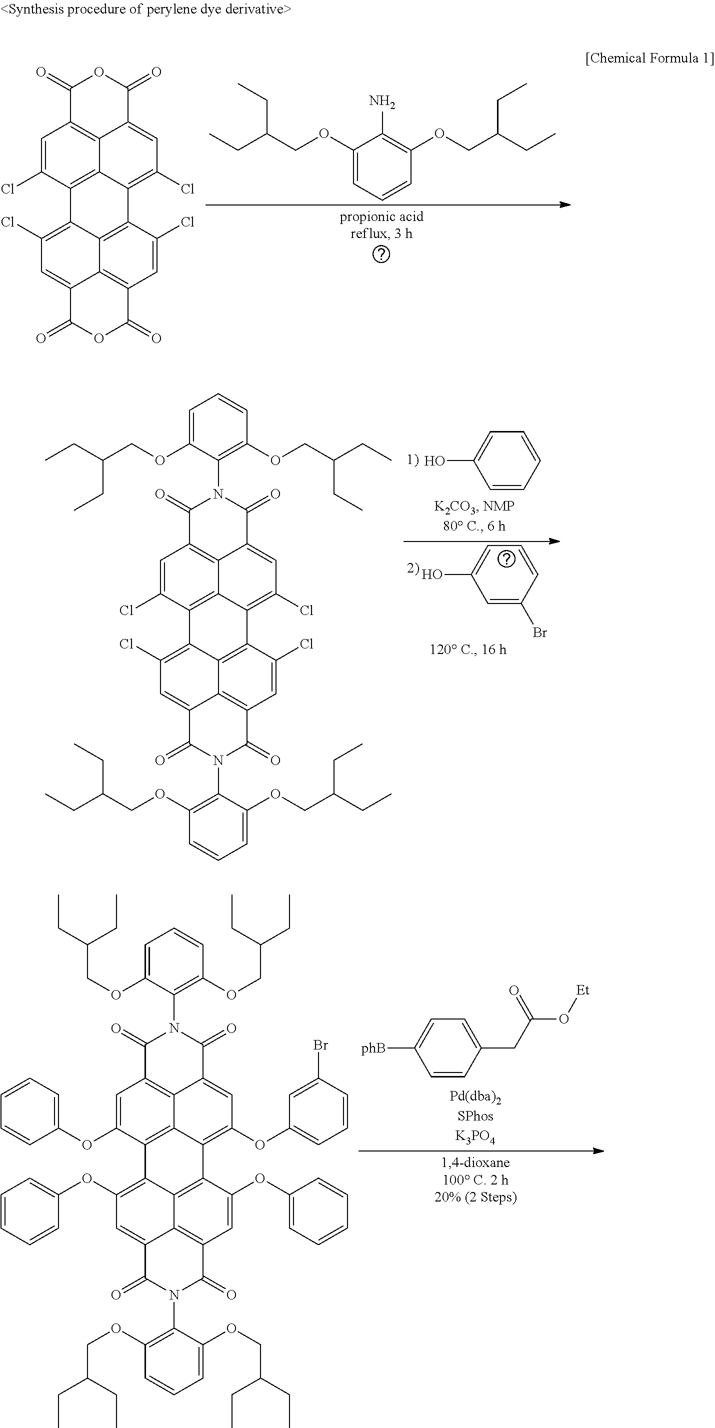

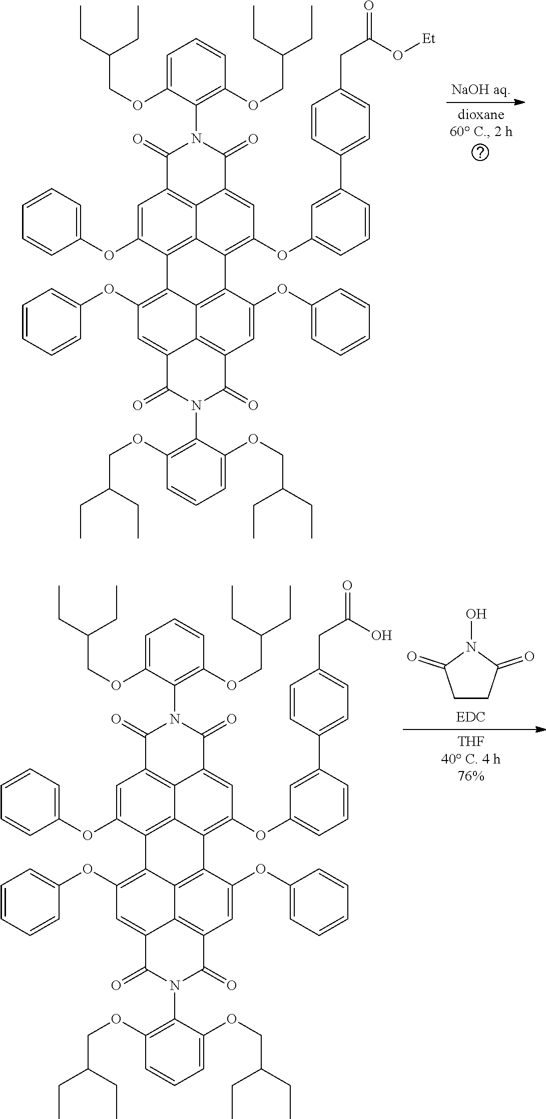

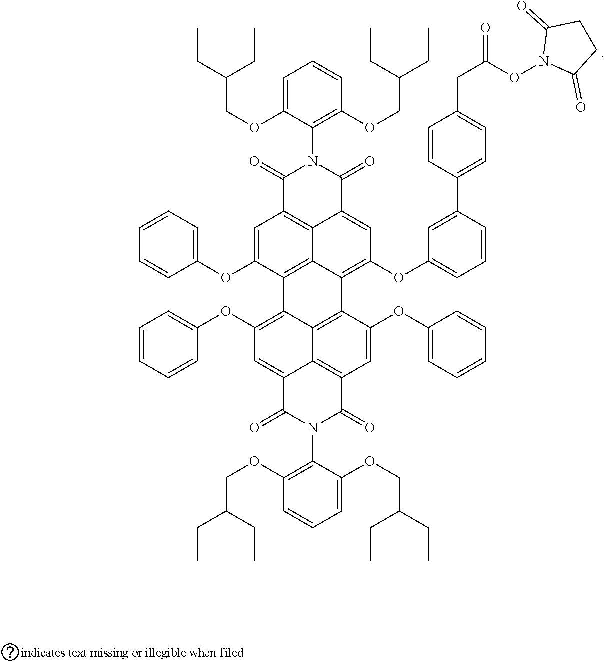 [SMILES: CCC(CC)COc1cccc(OCC(CC)CC)c1N1C(=O)c2cc(Oc3ccccc3)c3c4c(Oc5ccccc5)cc5c6c(cc(Oc7cccc(-c8ccc(CC(=O)ON9C(=O)CCC9=O)cc8)c7)c(c7c(Oc8ccccc8)cc(c2c37)C1=O)c64)C(=O)N(c1c(OCC(CC)CC)cccc1OCC(CC)CC)C5=O]

The perylene dye derivative synthesized as described above was dissolved in dimethylformamide (DMF) at 20 mg/ml. Thereafter, by adding 3-aminopropyltriethoxysilane (APS) (manufactured by Tokyo Chemical Industry Co., Ltd.) so that the molar ratio to the perylene dye derivative was 1, and causing reaction at room temperature for 1 hour, a triethoxysilyl group was added to the perylene dye derivative to obtain perylene-APS.

<Synthesis of Fluorescent Silica Nanoparticles>

747 μl of perylene-APS was added to a solution obtained by mixing 10844 μl of ethanol (99.5, manufactured by FUJIFILM Wako Pure Chemical Corporation), 909 μl of water, and 266 μl of aqueous ammonia (28%, manufactured by FUJIFILM Wako Pure Chemical Corporation), 234 μl of tetraethoxysilane (TEOS, manufactured by Tokyo Chemical Industry Co., Ltd.) diluted to 10% with ethanol placed in a microsyringe pump (SPS-1, manufactured by AS ONE Corporation) in advance was added thereto for 5 seconds, and the solution was stirred at room temperature for 48 hours. At this time, the total amount was 13 ml, and a molar ratio of the perylene dye and the tetraethoxysilane (perylene dye/tetraethoxysilane) was 10.

The reaction solution described above was centrifuged at 18000 G for 15 minutes in a cooled centrifuge (himac CR21N, manufactured by Koki Holdings Co., Ltd.), added with 13 ml of ethanol after removal of supernatant, and irradiated with ultrasonic waves to be redispersed. Washing by centrifugation, supernatant removal, and redispersion in ethanol was repeated three times. In this way, fluorescent silica nanoparticles of Example 1 were obtained.

### Examples 2 to 4

Fluorescent silica nanoparticles of Examples 2, 3, and 4 were individually obtained similarly to Example 1, except that the molar ratio of the perylene dye and the tetraethoxysilane (perylene dye/tetraethoxysilane) was changed to 15, 25, and 2.

### Examples 5 to 7

Fluorescent silica nanoparticles of Examples 5, 6, and 7 were individually obtained similarly to Example 1, except that the addition time of the tetraethoxysilane was changed to 70 hours, 48 hours, and 0.5 hours.

### Comparative Example 1

747 μl of perylene-APS was added to a solution obtained by mixing 10844 μl of ethanol (99.5, manufactured by FUJIFILM Wako Pure Chemical Corporation), 909 μl of water, and 266 μl of aqueous ammonia (28%, manufactured by FUJIFILM Wako Pure Chemical Corporation), tetraethoxysilane (164 μl, 70% of the total amount) was added at once, and the solution was stirred at room temperature for 3 hours. Thereafter, tetraethoxysilane (70 μl, 30% of the total amount) was further added at once, and the solution was stirred at room temperature for 48 hours. At this time, the total amount is 13 ml, and the molar ratio of the perylene dye and the tetraethoxysilane (perylene dye/tetraethoxysilane) is 10.

The reaction solution described above was centrifuged and washed similarly to Example 1, to obtain fluorescent silica nanoparticles of Comparative Example 1.

### Comparative Example 2

164 μl, which was 70% of the total amount of tetraethoxysilane, was added at once, and the solution was stirred at room temperature for 0.5 hours. Fluorescent silica nanoparticles of Comparative Example 2 were obtained similarly to Comparative Example 1 except that 70 μl, which was 30% of the total amount of tetraethoxysilane, was added at once thereafter.

### Comparative Example 3

Fluorescent silica nanoparticles of Comparative Example 3 were obtained similarly to Example 1 except that tetraethoxysilane was continuously added for 200 hours.

### Comparative Example 4

Comparative Example 4 was made similarly to Example 1 except that the molar ratio of the perylene dye and the tetraethoxysilane was changed to 50, but the amount of the perylene dye was too large to form particles.

### Comparative Example 5

Fluorescent silica nanoparticles of Comparative Example 5 were obtained similarly to Example 1 except that the molar ratio of the perylene dye and the tetraethoxysilane was changed to 0.5.

[Evaluation]

The fluorescent silica nanoparticles obtained as described above were evaluated as follows.

(Contained Amount of Dye)

For a contained amount of dye, amounts of Si and C elements were measured by the following procedure, an element ratio between Si and C was calculated. Thereafter, the Si ratio was converted into a molecular volume of SiO₂, the C ratio was converted into a molecular volume of the fluorescent dye, and a total volume (%) of the fluorescent dyes contained in the fluorescent silica nanoparticles was calculated. At this time, densities of SiO₂ and the fluorescent dye were 2.2 g/cm³ and 1.2 g/cm³, respectively.

<Quantification of Si>

Sulfuric acid was added to each fluorescent silica nanoparticle to cause ashing, and then lithium tetraborate was added to produce a bead. Quantification of Si was performed by a calibration curve method with a wavelength differential fluorescent X-ray analyzer (ZSX Primus IV manufactured by Rigaku Corporation).

<Quantification of C>

Each fluorescent silica nanoparticle was wrapped with a tin foil, and quantification of C was performed using a CHN elemental analyzer (vario EL cube manufactured by Elementer).

Measurement results are shown in Table 1 below.

(Emission Quantum Yield)

An emission quantum yield was measured using an absolute PL quantum yield measuring apparatus (Quantaurus-QY C11347-01 manufactured by Hamamatsu Photonics K.K.) by dispersing fluorescent nanoparticles in ethanol so that absorbance (abs.) was 0.2 to 0.4 at an excitation wavelength of 567 nm. Measurement results are shown in Table 1 below.

(Average Particle Diameter and Coefficient of Variation (CV Value))

For an average particle diameter of each silica nanoparticle obtained as described above, a diameter of each particle (100 pieces or more) shown in an image captured with a scanning electron microscope (manufactured by Hitachi High-Technologies Corporation, S-4800) was measured using image analysis software (A-zou Kun, manufactured by Asahi Kasei Engineering Corporation), and calculated as an average value thereof. A coefficient of variation was also calculated from the measured diameter. Measurement results are shown in Table 1 below.

(Luminance)

For luminance of each fluorescent silica nanoparticle obtained as described above, dilution was performed with ethanol using a spectrofluorometer F-7000 (manufactured by Hitachi High-Tech Science Corporation) so that a solid content concentration of the fluorescent nanoparticle dispersion was 0.028 mg/ml, a photomultiplier voltage was set to 400 V, and a fluorescence intensity at 610 nm in excitation light of 567 nm was measured. Measurement results are shown in Table 1 below.

(C/Si)

For C/Si, which is an atomic ratio of carbon to silicon on a surface of the fluorescent silica nanoparticles, a solid content concentration of the fluorescent nanoparticle dispersion was adjusted with ethanol to 0.028 mg/ml by using X-ray photoelectron spectroscopy (Quantera SXM manufactured by ULVAC-PHI, Inc.), 8 μl was dropped on a back surface of an aluminum foil previously wiped with ethanol, and a naturally dried film was observed. Measurement results are shown in Table 1 below.

TABLE 1

| | | | | | Evaluation | | | | | |
|---|---|---|---|---|---|---|---|---|---|---|
| | Manufacturing condition | | | | Contained | Emission | | Average | | |
| | Addition | Addition | | Fluorescent | amount of | quantum | CV | particle | | |
| | method of | time of | | dye/TEOS | dye | yield | value | diameter | | |
| | TEOS | TEOS | | (molar ratio) | (vol %) | (%) | (%) | (nm) | Luminance | C/Si |
| Example 1 | Continuous | 5 | seconds | 10 | 45 | 16 | 14 | 72 | 412 | 5.6 |
| Example 2 | Continuous | 5 | seconds | 15 | 55 | 11 | 19 | 76 | 320 | 8.6 |
| Example 3 | Continuous | 5 | seconds | 25 | 58 | 11 | 18 | 78 | 380 | 8.7 |
| Example 4 | Continuous | 5 | seconds | 2 | 10 | 35 | 8 | 77 | 90 | 2.1 |
| Example 5 | Continuous | 70 | hours | 10 | 47 | 22 | 19 | 240 | 610 | 4.3 |
| Example 6 | Continuous | 48 | hours | 10 | 45 | 18 | 18 | 212 | 484 | 4.8 |
| Example 7 | Continuous | 0.5 | hours | 10 | 45 | 16 | 16 | 95 | 420 | 5 |
| Comparative Example 1 | Divided | — | | 10 | 46 | 5 | 36 | 89 | 135 | 12 |
| Comparative Example 2 | Divided | — | | 10 | 47 | 4 | 38 | 89 | 135 | 12 |
| Comparative Example 3 | Continuous | 200 | hours | 10 | 2 | 25 | 32 | 320 | 43 | 0.3 |
| Comparative Example 4 | Continuous | 5 | seconds | 50 | Particle | — | — | — | — | — |
| | | | | | cannot be | | | | | |
| | | | | | formed | | | | | |
| Comparative Example 5 | Continuous | 5 | seconds | 0.5 | 2 | 36 | 8 | 80 | 41 | 0.5 |

When Examples 1 to 7 are compared with Comparative Examples 1 and 2, while TEOS was continuously added to the solution containing the fluorescent dyes in Example 1 to 7, TEOS was added in a divided manner in Comparative Examples 1 and 2. As a result, while the emission quantum yield was as high as 10% or more in Example 1 to 7, the emission quantum yield was as low as 5% and 4% in Comparative Examples 1 and 2. Further, while C/Si was in a range of 2 to 10 in Example 1 to 7, C/Si was as high as 12 in Comparative Examples 1 and 2.

This is considered to be because, in Example 1 to 7, TEOS, which is more likely to be hydrolyzed and polycondensed, is continuously added to the solution containing the fluorescent dyes, which are less likely to be hydrolyzed and polycondensed, and the fluorescent dye and the TEOS are bonded more, preventing the fluorescent dyes from being bonded to approximate each other. As a result, it is considered that, in the fluorescent silica nanoparticles of Example 1 to 7, concentration quenching is suppressed while the fluorescent dyes are contained at a high concentration, and the emission quantum yield is high. Whereas, in Comparative Examples 1 and 2, since the TEOS was added in a divided manner, it is considered that the bonding between the fluorescent dye and the TEOS was smaller, the fluorescent dyes were bonded to each other more, concentration quenching occurred, and the emission quantum yield becomes low.

When Examples 1 to 7 are compared with Comparative Example 3, while the addition time of the TEOS was set to 5 seconds to 70 hours in Example 1 to 7, it was set to 200 hours in Comparative Example 3. As a result, while a contained amount of dye was as high as 10 vol % or more in Example 1 to 7, it was as low as 2 in Comparative Example 3. This is considered to be because, in Example 1 to 7, while TEOS, which is more likely to be hydrolyzed and polycondensed, is continuously added to the solution containing the fluorescent dyes, which are less likely to be hydrolyzed and polycondensed, and the fluorescent dye and the TEOS were more bonded to each other, the bonding with the TEOS was insufficient in Comparative Example 3 since the addition time of the TEOS was too long and the bonding between the fluorescent dyes increased.

When Examples 1 to 7 are compared with Comparative Example 4, the molar ratio of the fluorescent dye/TEOS is as high as 40 in Comparative Example 4. As a result, in Comparative Example 4, the amount of the fluorescent dyes was too large to form particles.

When Examples 1, 5, 6, and 7 are individually compared, the fluorescent dye/TEOS is 10 in all of these, but the continuous addition time of the TEOS became shorter in the order of Examples 5, 6, 7, and 1. As a result, in this order, the particle diameter of the fluorescent nanoparticles became smaller, and the coefficient of variation (CV value) became lower.

This application claims priority based on Japanese Patent Application No. 2020 016145 filed on Feb. 3, 2020. The contents described in the specification and drawings of this application are all incorporated herein by

### INDUSTRIAL APPLICABILITY

The fluorescent silica nanoparticles according to the present embodiment have high luminance, and thus are useful for fluorescence imaging and the like.

1. Fluorescent silica nanoparticles comprising:
silica nanoparticles; and
fluorescent dyes contained in the silica nanoparticles, wherein a total volume of the fluorescent dyes is 5% or more with respect to a total volume of the fluorescent silica nanoparticles, and an emission quantum yield of the fluorescent silica nanoparticles is 10% or more.

2. The fluorescent silica nanoparticles according to claim 1, wherein an atomic number ratio (C/Si) of carbon to silicon on a surface of the fluorescent silica nanoparticles as measured by X-ray photoelectron spectroscopy is 2 to 10.

3. The fluorescent silica nanoparticles according to claim 1, wherein a coefficient of variation in particle diameter of the fluorescent silica nanoparticles is 20% or less.

4. The fluorescent silica nanoparticles of claim 1, wherein the fluorescent silica nanoparticles have an average particle diameter of 5 to 250 nm.

5. A method for manufacturing the fluorescent silica nanoparticles according to claim 1, the method comprising:
continuously adding alkoxysilane to liquid containing fluorescent dyes, ammonia, and water so that a molar ratio of the fluorescent dyes to the alkoxysilane is in a range of 1 to 30.

6. The method for manufacturing fluorescent silica nanoparticles according to claim 5, wherein a continuous addition time of the alkoxysilane is 72 hours or less.

7. The method for manufacturing fluorescent silica nanoparticles according to claim 5, wherein a continuous addition time of the alkoxysilane is 1 hour or less.

8. The fluorescent silica nanoparticles according to claim 2, wherein a coefficient of variation in particle diameter of the fluorescent silica nanoparticles is 20% or less.

9. The fluorescent silica nanoparticles of claim 2, wherein the fluorescent silica nanoparticles have an average particle diameter of 5 to 250 nm.

10. The fluorescent silica nanoparticles of claim 3, wherein the fluorescent silica nanoparticles have an average particle diameter of 5 to 250 nm.
